(19) United States
(12) Patent Application Publication
(10) Pub. No.: US 2017/0339984 A1
(43) Pub. Date: Nov. 30, 2017

(54) USE OF D-RIBOSE TO ENHANCE ADAPTATION TO PHYSICAL STRESS

(71) Applicant: Bioenergy Life Science, Inc., Ham Lake, MN (US)

(72) Inventor: Yongquan Xue, Ham Lake, MN (US)

(21) Appl. No.: 15/524,235

(22) PCT Filed: Nov. 3, 2015
(86) PCT No.: PCT/US15/58902

§ 371 Date: May 3, 2017

Related U.S. Application Data
(60) Provisional application No. 62/074,611, filed on Nov. 3, 2014.

(51) Int. Cl. A23L 2/52; A23L 33/125; A61K 31/7004; A61K 9/00; A61K 9/08; A23L 2/38

(52) U.S. Cl. CPC A23L 2/52; A61K 31/7004; A61K 9/0056; A23L 33/125; A23L 2/38; A61K 9/08

(57) ABSTRACT

Methods of improving adaptation to physical stress by administering D-ribose and methods of administering D-ribose to improve adaptation to physical exercise.

### BACKGROUND

Physical stress such as heavy work or a new exercise regime causes tissues strains or damage. These strains or damages triggers changes to occur in the tissue, a process called physical adaptation. Physiological adaptations start to occur almost immediately when beginning a new exercise program. It is very important for a successful training and eventual physical performance. Especially for beginners or for people who are unfit or do not engage in regular exercise, physical adaptation could be a long a painful process, which can lead to a high dropout rate. Thus, the physical adaptation to exercise is more of a challenge with unfit individuals. It is therefore desirable to find ways to alleviate the pain associated with beginning a new exercise regime and enhancing adaptation to physical stress.

Through experimentation, it has been discovered that D-ribose enhances adaptation to physical exercise.

### DESCRIPTION OF THE DRAWINGS

FIG. 1 depicts a bar representation of the rate of perceived exertion following exercise.

### DETAILED DESCRIPTION OF THE INVENTION

A high-intensity exercise protocol was designed as a double-blind, crossover study to assess the influence of D-Ribose adaptation to physical stress. Specifically, D-Ribose and a control (Dextrose) were administered on separate subjects at a dosage often grams per day (10 g/day). A variety of physiological parameters were measured in the subjects administered D-Ribose (DR) supplementation (i.e., the DR subjects) versus the subjects administered Dextrose (DEX) supplementation (i.e., the DEX subjects).

### Study Methodology

The subjects consisted of twenty-six (26) healthy individual (10 females, 16 males). Each subject was randomly categorized as a DR subject or a DEX subject for the administration of supplementation. Furthermore, each subject was required to maintain his or her normal diet during the study, as well as performing his or her normal daily activities without performing any additional separate exercise sessions not part of the study protocol.

To test D-ribose for adaptation, the twenty-six (26) adult subjects were further divided into two subgroups based on their fitness level (i.e., peak oxygen uptake (VO₂ max) results. The first subgroup comprised subjects with higher VO₂ max results (i.e., the “Fit Subgroup”) and the second subgroup comprised subjects with lower VO₂ max results (i.e., the “Unfit Subgroup”). The Unfit Subgroup consisted of six (6) females and seven (7) males. The average age of the Unfit Subgroup was 27.7±3.4 years and the average peak VO₂ of the Unfit Subgroup was 39.9±4.1 mL/kg/min. The Fit Subgroup consisted of four (4) females and nine (9) males. The average age of the Fit Subgroup was 27.6±3.5 year and the average peak VO₂ of the Fit Subgroup was 52.2±4.3 mL/kg/min.

On the load days (i.e., the two (2) days prior to the exercise sessions), DR subjects consumed five grams (5 g) of DR mixed with either their food or in a self-selected beverage with lunch and an additional five grams (5 g) with dinner (i.e., between three to eight hours apart), while DEX subjects consumed five grams (5 g) of DEX mixed with either their food or in a self-selected beverage with lunch and an additional five grams (5 g) with dinner (i.e., between three to eight hours apart).

On the exercise session days (i.e., three (3) days following the load days), DR subjects ingested a standardized pre-exercise snack containing five grams (5 g) of DR at two (2) hours before the exercise session and five grams (5 g) of DR following the exercise session but before leaving the laboratory (i.e., within one hour following the exercise session), while DEX subjects ingested a standardized pre-exercise snack containing five grams (5 g) of DEX at two (2) hours before the exercise session and five grams (5 g) of DEX following the exercise session but before leaving the laboratory (i.e., within one hour following the exercise session). For both DR subjects and DEX subjects, the standardized snacks were self-selected but were based on the subjects' normal dietary habits. The snacks were consistent from day to day and consisted of one hundred seventy grams (170 g) of yogurt and two granola bars, along with the designated supplement. Subjects were asked to record their diets so that there would be consistency throughout the testing period. Following an exercise session, each subject ingested the final daily dose of five grams (5 g) before leaving the laboratory. Subjects also ingested two hundred milliliters (200 ml) of water at twenty (20) and forty (40) minutes of exercise to minimize the effects of dehydration, which can occur during periods of high-intensity exercise.

The protocol of the double-blind crossover study involved an initial baseline assessment, followed by two separate day assessments after consuming either a DR or DEX supplement. Each exercise session entailed measurements of creatine kinase (CK), blood urea nitrogen (BUN), glucose, heart rate (HR), rate of perceived exertion (RPE), and power output (PO) measurements.

### Experiment Design

Pre-Testing (Baseline) Assessment

During each subject's first visit to the laboratory, the subject underwent a maximal oxygen uptake and blood lactate evaluation and practiced the two-minute power test assessment using a cycle ergometer. Initially using the cycle ergometer, each subject completed a warm-up exercise for five minutes at a self-selected cadence at one kilogram (1 kg) resistance. Cycling resistance was then increased at a rate of one-half kilogram per four-minute interval (0.5 kg/4 min) until volitional exhaustion. Heart rate (HR), oxygen uptake (VO₂) and a blood lactate sample was collected at the three-minute, thirty-second (3′30″) mark and four-minute (4′) mark of each stage. This assessment established exercise workloads during the subsequent two (2) treatment sessions.

Treatment Assessments

Each subject was randomly assigned to be a DR subject (for administration of DR supplementation) or a DEX subject (for administration of DEX supplementation). Apart from the supplementation provided to and consumed by the subject, the treatment protocols were identical. The specific treatment protocol (i.e., administration of supplementation and exercise sessions) is detailed in Table 1 below:

TABLE 1
Treatment Protocol

| Day | Action Performed |
|---|---|
| 1. | 2x treatment dosage of 5 g of supplement |
| | (DR or DEX); no exercise session |
| 2. | 2x treatment dosage of 5 g of supplement |
| | (DR or DEX); no exercise session |
| 3. | 2x treatment dosage of 5 g of supplement |
| | (DR or DEX) + 1 exercise session |
| 4. | 2x treatment dosage of 5 g of supplement |
| | (DR or DEX) + 1 exercise session |
| 5. | 2x treatment dosage of 5 g of supplement |
| | (DR or DEX) + 1 exercise session |

Each exercise session consisted of six (6) ten-minute intervals of exercise on a cycle ergometer. During each ten-minute interval, the subject cycled for eight (8) minutes at a workload of approximately 60% of the subject's VO₂ max, then immediately cycled for an additional two (2) minutes at a workload of approximately 80% VO2 max (approximately one workload above the subject's calculated lactate threshold). Cadence and power output were monitored at ten-minute intervals during each exercise session. At the end of the sixty-minute exercise session, each subject completed a two-minute performance task (time trial). This performance task required the subject to produce as much power as possible during the two-minute interval. Peak power, average power, and percent decline were assessed during this two-minute task trial. Workload for performance task was set at five percent (5%) of the subject's body weight.

Physiologic parameters were measured and hydration was provided to the subjects during the exercise session. The same protocol for testing and hydration protocol was followed for both DR subjects and DEX subjects. Blood samples were drawn from each subject via a venipuncture technique at the following time periods:
Ten (10) minutes before commencement of exercise;
Twenty (20) minutes after the commencement of and during exercise;
Forty (40) minutes after the commencement of and during exercise;
Sixty (60) minutes after the commencement of and during exercise; and
Twenty-four (24) hours after the conclusion of exercise (twenty-five (25) hours after the commencement of exercise).

Blood glucose was measured at all of the above time points except at twenty-four (24) hours post-exercise. Creatine kinase and BUN levels were measured at the pre-exercise (−10 min.) point during the three (3) days of exercise and twenty-four (24) hours post-exercise following the third (last) exercise session.

A “Rating of Perceived Exertion” (RPE) was recorded every twenty (20) minutes during exercise using the Borg 1-10 scale. The Likert scale (0-10 points) was used to subjectively assess quadriceps muscle soreness, overall fatigue, appetite, perceived performance, and sleep quality. These scales were completed prior to and following each exercise session.

The treatment testing and hydration protocol is summarized in Table 2 below:

TABLE 2
Testing and Hydration Protocol

| | Timeframe (Commencement of Exercise) | | | | | | |
|---|---|---|---|---|---|---|---|
| Measurement/ | −10 | | 20 | 40 | 60 | 65 | 25 |
| Activity* | min. | Start** | min. | min. | min. | min. | hr. |
| Likert | X | — | — | — | — | X | — |
| RPE | — | — | X | X | X | — | — |
| Blood | X | — | — | — | X | — | X |
| Power Test | — | — | — | — | — | X | — |
| Drink | — | — | X | X | — | — | — |

*“X” indicates that the measurement was taken or activity (i.e., hydration) performed; “—” indications that the measurement was not taken or the activity was not performed.
**Indicates the start of the sixty-minute exercise session.

Instrumental Assessment

Heart rate was recorded using a Polar HR monitor. Blood glucose levels were measured using a Bayer glucose monitor. Blood lactate levels were measured by an AccuSport Lactate Analyzer. Creatine kinase and BUN were measured utilizing an Abaxis Piccolo analyzer. Power data from the time trial performance test was assessed with the Sports Medicine Industries (SMI) software package.

### Statistical Analysis

All tabulated data was analyzed with StatPac and SPSS statistical software using a 2-way ANOVA with repeated measures, time and treatments as independent variables. A Turkey's post hoc test was used to differentiate means if a significant interaction was observed. Heart rate, RPE, serum lactate, levels, serum CK levels, serum BUN levels and measured power data were dependent measures. An alpha level of significance was set at p<0.05.

### Results

All twenty-six (26) subjects completed the study without any adverse events. The DR subjects and the DEX subjects tolerated their respective supplements without any subjective complaints or issues. Data are presented as main effects as there were no interactions.

The Unfit and Fit Subgroups were established as shown in Table 3 below:

TABLE 3
Unfit max/Fit Subgroup Classification Based Upon Performance Data*

| Measurement | Unfit DR | Unfit DEX | Fit DR | Fit DEX |
|---|---|---|---|---|
| Mean Power | 0.20 ± 0.32 | −0.09 ± 0.31 # | 0.08 ± 0.31 | 0.07 ± 0.33 |
| (W/kg BW)** | | | | |
| CK (u)** | 10.3 ± 79.3 | 124.5 ± 126.5 # | 40.0 ± 348.1 | 12.1 ± 270 |
| HR (bpm)*** | 151.5 ± 19.4 | 152.0 ± 19.5 | 152.6 ± 11.9 | 152.4 ± 11.7 |
| RPE (Borg 6-20 | 13.1 ± 1.6 | 13.5 ± 1.5 # | 13.6 ± 1.8 | 13.9 ± 1.6 |
| scale)**** | | | | |

*Data are mean ± SD
**Mean power reflects the difference between day 1 and day 3 per treatment
***Creatine kinase levels, Day 1 to Day 3
# Significance between DR versus DEX

Relative and absolute mean power data can be found in Table 4 below:

TABLE 4
Relative and Absolute Mean Power Output Changes*

| Supplement | Measurement | Unfit Subgroup | | Fit Subgroup | |
|---|---|---|---|---|---|
| Ribose | Relative | 0.17 (0.32) | W/kg BW ** | 0.08 (0.39) | W/kg BW |
| | Absolute | 13.2 (24.2) | W *** | 2.3 (17.1) | W |
| Dextrose | Relative | −0.09 (0.29) | W/kg BW | 0.07 (0.33) | W/kg BW |
| | Absolute | −8.9 (22.4) | W | 8.2 (27.7) | W |

*Mean (+SD)
** Significantly different from Dextrose (p = 0.04)
*** Significantly different from Dextrose (p = 0.01)

D-ribose ingestion led to a significant (p=0.04) improvement in relative mean power of 288% over DEX in the Unfit Subgroup. There was also a significant difference between DR and DEX in the change of absolute mean power of 245% (p=0.01) for this subgroup. A significant difference between DR and DEX was found for relative (p=0.05) and absolute (p=0.02) peak power output for the Unfit Subgroup. The average changes in relative and absolute peak power from Day 1 to Day 3 were 0.33+0.52 W/kg BW and 26.8±40.8 W for DR while DEX were −0.09+0.51 W/kg BW and −10.8±33.0 W, respectively.

Relative and absolute mean power outputs were not different between DR and DEX treatments for the Fit Subgroup. No differences between treatments were noted for relative (p=0.27) and absolute (p=0.79) peak power for the Fit Subgroup. The average changes in relative and absolute peak power from Day 1 to Day 3 were 0.15±0.41 W/kg BW and 6.2±28.6 W for DR while DEX were −0.02+0.37 W/kg BW and 3.31±25.8 W, respectively.

Analysis of serum CK data indicated that DR ingestion led to lower change for the Unfit Subgroup. Creatine kinase levels increased by an average of 37.1±85.2 U for the DR treatment compared to the DEX treatment of 121.4±110.2 U (p=0.03). No statistical difference (p=0.88) was observed for change in BUN levels between DR (0.93±2.66) and DEX (1.08±2.56) treatments for the Unfit Subgroup. No differences for change in CK and BUN levels were observed between DR and DEX treatments in the Fit Subgroup. As noted in Table 5 below, no differences were observed for blood glucose and remained stable for all treatments and within both subgroups:

TABLE 5
Blood Glucose Levels During Exercise*

| | Unfit Subgroup | | | Fit Subgroup | | |
|---|---|---|---|---|---|---|
| Supplement | 19.5 | 39.5 | 59.5 | 19.5 | 39.5 | 59.5 |
| Ribose | 4.0 (0.6) | 4.0 (0.6) | 4.1 (0.7) | 3.8 (0.5) | 4.0 (0.5) | 3.9 (0.5) |
| Dextrose | 4.0 (0.5) | 4.0 (0.5) | 3.9 (0.6) | 4.0 (0.6) | 4.1 (0.7) | 4.0 (0.6) |

*Mean (+ SD); values in mM/L

No difference between treatments was found for HR in the Unfit Subgroup. Average HR for the DR trial was 152±20 bpm and 153±17 bpm for the DEX trial. The RPE was significantly lower (p=0.003) for DR (13±2) than DEX (14±2). Average HR and RPE were not different between DR and DEX for the Fit Subgroup, 153±12 bpm and 14±2 versus 153±12 bpm and 14±2, respectively.

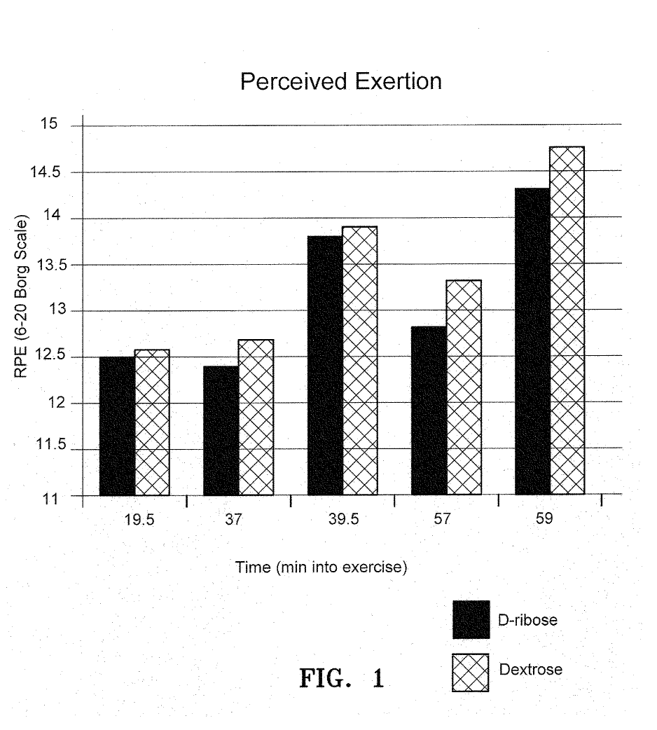

As depicted in FIG. 1, the average rate of perceived exertion was greater for DEX subjects than the average rate of perceived exertion for DR subjects at all measured points of the exercise sessions.

The potential beneficial role of DR depends upon the type, degree of intensity and duration of exercise, and also on the fitness level of the subject. Performance was evaluated for subjects administered DR or DEX orally around high-intensity exercise. From Day 1 to Day 3, mean and peak power increased significantly in DR subjects in the Unfit Subgroup as compared to DEX subjects in the Unfit Subgroup. Mean and peak power between was maintained by DR subjects and DEX subjects in the Fit Subgroup. Furthermore, RPE was significantly lower in the DR subjects than for the DEX subjects.

Multiple factors can account for the benefits with DR, including changes in serum chemistry markers, such as CK, BUN, and glucose levels. For example, differences in muscular CK levels might have shed light on this beneficial difference by indicating a maintenance, or lack thereof, of cell membrane integrity. The change in CK level from Day 1 to Day 3 was about three times (3×) greater for the DEX treatment as compared to DR in the Unfit Subgroup.

Similar results have also been found with administering DR at lower dosages of six grams per day (6 g/day) to subjects. Wherein on the load days (i.e., the two (2) days prior to the exercise sessions), three grams (3 g) of DR was mixed with either their food or in a self-selected beverage with lunch and an additional three grams (3 g) with dinner and on the exercise session days (i.e., three (3) days following the load days), the subjects ingested a standardized pre-exercise snack containing three grams (3 g) of DR at two (2) hours before the exercise session and three grams (3 g) of DR following the exercise session within one hour following the exercise session.

The delivery and utilization of oxygen to exercising muscle is a major factor in assessing fitness and VO₂ max levels. Separating the data for lower and higher VO₂ max subgroups reveals significant differences in relation to the effect of DR during high-intensity exercise. Specifically, the Unfit Subgroup of DEX subjects had a significant increase in CK levels by more than three-fold and a greater RPE, as compared to the Unfit Subgroup of DR subjects. Furthermore, in the Unfit Subgroup, the subjects improved their power test output. This suggests that individuals that have not consistently performed exercise above the lactate threshold level do not fair equally with individuals that exercise or train on a more intense regimen schedule, even on a relative basis. The rise in CK levels observed in the Unfit Subgroup appears to imply that a strenuous, anaerobic exercise of these muscle groups produced cellular stress in which enzymatic leaking occurs, which can not only effect cellular homeostasis, but exercise performance and potentially limit future scheduled bouts of exercise due to subjective symptoms.

In summary, D-ribose ingestion led to greater performance changes than DEX over three days of cycling. More importantly, when the group was subdivided into unfit and fit groups, within and between group differences were accentuated. The unfit (lower VO₂ max) group benefited from DR ingestion and was able to maintain performance for the next day's work. Biochemical analysis revealed that there was less muscle damage with DR ingestion compared to DEX. Therefore, it is concluded that D-ribose enhances adaptation to physical stress, which leads to better performance in the end.

1. A method of enhancing adaptation to physical exercise of humans comprising oral administration of D-ribose prior to a period of physical exercise and oral administration of D-ribose during the period of physical exercise, wherein said subject demonstrates improved adaptation to physical exercise.

2. The method of claim 1 wherein the oral administration of D-ribose is about 6 to 10 grams per day prior to a period of physical exercise and about 6 to 10 grams of D-ribose a day during the period of physical exercise.

3. The method of claim 2 wherein the oral administration of D-ribose prior to the period of physical exercise is at least 2 days prior to the period of physical exercise.

4. The method of claim 3 wherein the oral administration of D-ribose comprises about 3 to 5 grams twice a day prior to the period of physical exercise and about 3 to 5 grams twice a day during the period of physical exercise.

5. The method of claim 4, wherein the about 3 to 5 grams of D-ribose twice a day prior to the period of exercise are administered about 3 to 8 hours apart.

6. The method of claim 5, wherein the about 3 to 5 grams of D-ribose during the period of physical exercise is at least 2 hours before physical exercise and within 1 hour after physical exercise.

7. A method of claim 1 wherein the human has a lower exercise heart rate and lower rate of perceived exertion during physical exercise.

8. The method of claim 1 wherein the human has a low VO₂ max level.
